(19) United States
(12) Patent Application Publication
(10) Pub. No.: US 2024/0225703 A1
(43) Pub. Date: Jul. 11, 2024

(54) ANTIBIOTIC INTRAMEDULARY NAIL APPARATUS, SYSTEM, AND METHOD

(71) Applicant: Joseph Hoegler, Birmingham, MI (US)

(72) Inventor: Joseph Hoegler, Birmingham, MI (US)

(21) Appl. No.: 18/407,435

(22) Filed: Jan. 8, 2024

Related U.S. Application Data
(60) Provisional application No. 63/437,479, filed on Jan. 6, 2023.

(51) Int. Cl. A61B 17/72; A61B 17/00

(52) U.S. Cl. CPC A61B 17/72; A61B 2017/00464; A61B 2017/00889

(57) ABSTRACT

An apparatus that can include a threaded handle and a removable sleeve for inserting concrete.

### RELATED APPLICATIONS

This utility patent application claims priority to the following provisional patent application which is hereby incorporated by reference in its entirety: “ANTIBIOTIC INTRADMEDULLARY NAIL” (Ser. No. 63/437,479) that was filed on Jan. 6, 2023.

### BACKGROUND OF THE INVENTION

The invention relates generally to intramedullary nails. More specifically, the invention is an antibiotic intermedullary nail apparatus, system, and method (collectively, the “system”).

An open or compound fracture of a bone is a fracture in which there is an open wound or break in the skin near the site of the broken bone. Such fractures present an increased risk for infection relative to other types of fractures. While rates of infection can vary based on a variety of potential factors such as the severity of the fracture, the location of the fracture, and a long list patient-specific factors, the risk of an infection resulting from the fracture is a significant risk factor for a negative patient outcomes.

One way in which infections resulting from open fractures are treated, especially as it relates to long bones such as the tibia, is by inserting an antibiotic intramedullary nail (IMN) or rod into the medullary cavity of the fractured bone. An antibiotic IMN delivers local antibiotics directly to the bone and area surrounding the fracture (e.g., at the site of infection, in the event an infection is present) as well as provides added stability to the fracture. To that end, an antibiotic. IMN generally comprises a rigid, metal IMN having a first end and a second end opposite the first end wherein at least a portion of the IMN between the first and second ends is coated with an antibiotic cement such that an outer surface of at least a portion of the antibiotic IMN comprises antibiotic cement.

While conventional antibiotic IMNs have proven useful in delivering antibiotics and adding stability to a fractured bone, they are not without their drawbacks. For example, antibiotic IMNs are intended to be removed from the patient's body after a certain amount of time because bacteria can adhere to the IMN and become a source of new or continued infection. During the removal of conventional antibiotic IMNs, the cement coating may break off from the IMN and may be left in the medullary cavity of the patient's bone due at least in part to the cement covered portion of the IMN having a cross-sectional profile (e.g. size and/or shape) and/or cross-sectional area that is larger than other portions of the IMN (e.g., the cement-covered portion having a larger diameter than some or all of the rest of the IMN).

There are intramedullary nails approved for use in other countries that have a gentamicin coating (not cement). Gentamicin is an antibiotic used to treat gram negative bacterial infections. The most common cause of osteomyelitis is from gram positive bacteria. This antibiotic nail design allow for the cement to be made with antibiotics that cover gram positive and gram negative bacteria.

### SUMMARY OF THE INVENTION

The invention relates generally to intramedullary nails. More specifically, the invention is an antibiotic intermedullary nail apparatus, system, and method (collectively, the “system”).

An ultramedullary rod, which is also known as an ultramedullary nail (IMN) is a metal rod forced into the medullary cavity of a bone. IM nales have been used to treat fractures of long bones in the body going back to the treatment of soldiers with fractures of the femure back in 1939.

The IMN apparatus is easy to make and utilize compared to current IMN devices. The IMN apparatus can be easily inserted and removed from the bone through the use of a threaded insertion handle. The apparatus can stabilizing the fractured bone through the use of interlocking screws at the proximal and distal ends of the IMN apparatus. No fragmenting of cement when removing from bone do to smooth transition from metal part of nail to antibiotic cement portion. Enables the ability to customize antibiotics used in cement.

Once antibiotic nail is made it can be inserted into long bones to treat acute or chronic bone infections (osteomyelitis). It can also be used to treat acute fractures at high risk for infection while simultaneously providing prophylactic antibiotics. Can be use in the treatment of nonunions while ruling out infection or as definitive treatment of nonunions.

### BRIEF DESCRIPTION OF DRAWINGS

Different examples of various attributes, components, and configurations that can be incorporated into the system are illustrated in the drawings described briefly below. No patent application can expressly disclose in words or in drawings, all of the potential embodiments of an invention. In accordance with the provisions of the patent statutes, the principles, functions, and modes of operation of the system are illustrated in certain preferred embodiments. However, it must be understood that the system may be practiced otherwise than is specifically illustrated without departing from its spirit or scope.

FIG. 1 is a perspective diagram illustrating an example of an antibiotic intramedullary nail apparatus.

FIG. 2 is a side view diagram illustrating an example of an antibiotic intramedullary nail apparatus.

FIG. 3 is a cross-section view diagram illustrating an example of an antibiotic intramedullary nail apparatus.

FIG. 4 is a cross-section view diagram illustrating an example of an antibiotic intramedullary nail apparatus.

FIG. 5 is a cross-section view diagram illustrating an example of an antibiotic intramedullary nail apparatus.

FIG. 6 is a side view diagram illustrating an example of an antibiotic intramedullary nail apparatus.

FIG. 7 is a cross-section view diagram illustrating an example of an antibiotic intramedullary nail apparatus.

FIG. 8 is a perspective diagram illustrating an example of an antibiotic intramedullary nail apparatus.

FIG. 9 is a perspective diagram illustrating an example of a

FIG. 10 is a cross-section view diagram illustrating an example of an antibiotic intramedullary nail apparatus.

FIG. 11 is a top view diagram illustrating an example of a IMN apparatus with a removably threaded insertion handle and a cement opening in the sleeve.

FIG. 12 is a top view diagram illustrating an example of the IMN apparatus of FIG. 11 with a cement gun connected to the spout opening.

FIG. 13 is a flow chart diagram illustrating an example of a method for applying antibiotic intramedullary nail apparatus.

FIG. 14 is an environmental diagram illustrating an example of a system for applying an antibiotic intramedullary nail apparatus.

### DETAILED DESCRIPTION OF THE INVENTION

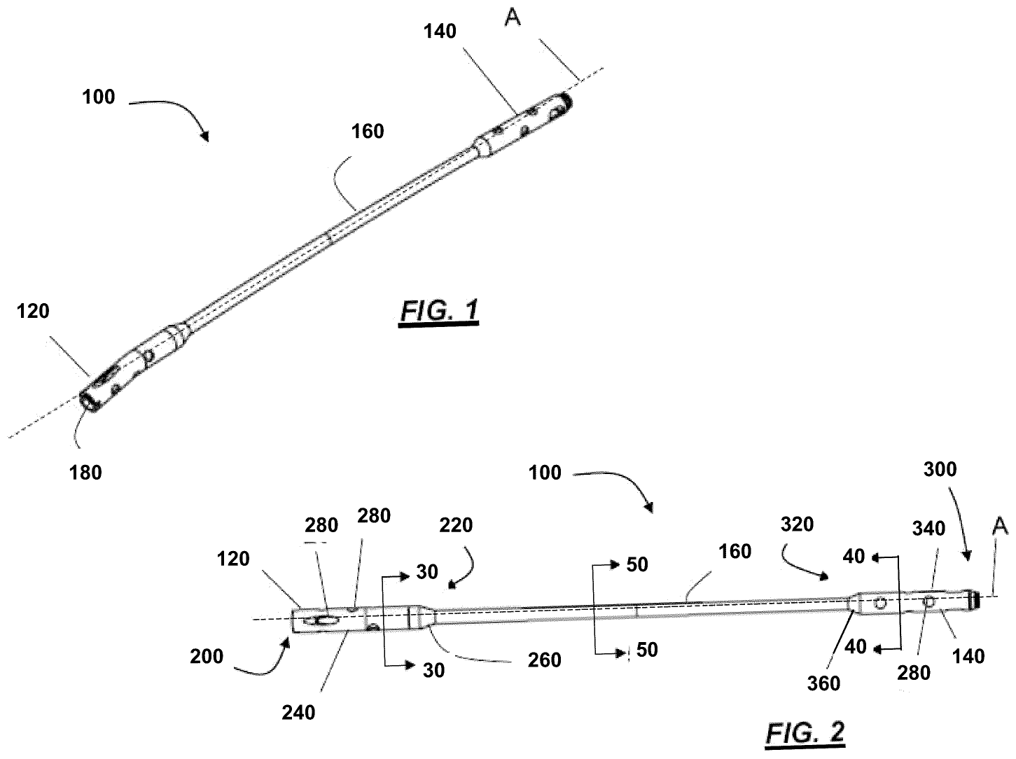

Referring in more detail to the drawings, FIGS. 1 and 2 depict an illustrative embodiment of an IMN 100 having a first end portion 120, a second end portion 140, an intermediate portion 160 disposed and extending between the first and second end portions 120, 140, and a longitudinal axis A that extends along and through the intermediate portion 160 between the first and second end portions 120, 140. Further, in at least some embodiments, the IMN 100 has a channel or passageway 180 therein (shown in FIG. 1) that extends through the entirety of the IMN 100 (i.e., through each of the first end portion 120, second end portion 140, and intermediate portion 160) configured to allow, for example, a ball-tipped wire to pass through the IMN 100.

With reference to FIG. 2, the first end portion 120 is located at the opposite end of the IMN 100 from the second portion 140 and is spaced apart from the second portion 140 by the intermediate portion 160. The first end portion 140 has a first end 200 and a second end 220, wherein the second end 220 is adjacent to the intermediate portion 160 of the IMN 100, and the first end 200 is spaced from the intermediate portion 16.

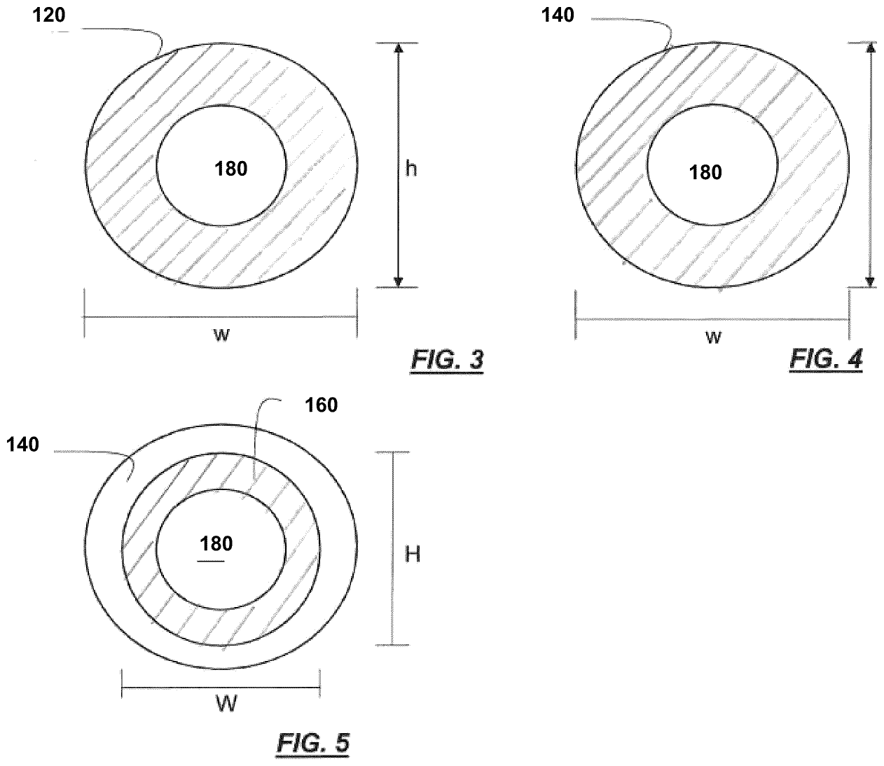

The first end portion 120 has a cross-sectional profile (e.g., shape and size (for example, one or more dimensions that extend perpendicular to the longitudinal axis A, such as height and width)) and cross-sectional area (e.g., cross-sectional surface area) taken in a plane that is perpendicular to the longitudinal axis A (see, for example, FIG. 3 showing the cross-sectional area of the first end portion and a height (h) and a width (w) dimension). In an embodiment, the cross-sectional profile and/or cross-sectional area of the first end portion 120 is constant as the first end portion 120 extends from the first end 200 to the second end 220. In other embodiments, however, different parts or sections of the first end portion 120 may have different cross-sectional profiles and/or cross-sectional areas. For example, a first part 240 of the first end portion 120 extending from the first end 200 thereof towards the second end 220 thereof has a constant cross-sectional profile and cross-sectional area, whereas a second part 260 of the first end portion 120 may have a smaller cross-sectional profile and/or cross-sectional area. In one particular example, such as, for example, that shown in FIG. 2, the second part 260 of the first end portion 120 may taper as it extends from the first part 240 towards the second end 220 of the first end portion 120 (i.e., towards the intermediate portion 160 of the IMN 100) such that the cross-sectional profile and cross-sectional area of the first end portion 120 gets progressively smaller as the second part 260 extends toward the second end 220 of the first end portion 120. Accordingly, it will be appreciated that the first end portion 120 may have a single cross-sectional profile and/or cross-sectional area or may have two or more parts or sections thereof that have different cross-sectional profiles and/or cross-sectional areas.

By way of illustration, however, in the embodiment illustrated in FIGS. 1 and 2, the first end portion 120 of the IMN 10 has a cross-sectional profile that is circular in shape, as is shown in FIG. 3, and has a cross-sectional area also shown in FIG. 3. In such an embodiment, the first end portion 120 may have a single, constant diameter from the first end 200 to the second end 220 (e.g., a diameter of 9 mm, in an embodiment). In other embodiments, however, different parts or sections of the first end portion 120 may have different outer diameters. For example, in the illustrative embodiment shown in FIG. 2, the first part 240 of the first end portion 120 extending from the first end 200 thereof towards the second end 220 thereof has a constant diameter, whereas the second part 260 of the first end portion 120 tapers as it extends from the first part 240 towards the second end 220 of the first end portion 120 (i.e., towards the intermediate portion 160 of the IMN 100) such that the outer diameter gets progressively smaller as the second part 260 extends toward the second end 220 of the first end portion 120. Accordingly, it will be appreciated that in an embodiment wherein the first end portion 120 has a circular cross-sectional profile, the first end portion 120 may have a single constant outer diameter or may have two or more parts or sections thereof that have different outer diameters.

In addition to the above, and as shown in, for example, FIG. 2, the first end portion 120 may include one or more apertures or holes 280 therein configured to receive locking screws and through which locking screws may pass to allow the first end portion 120 of the IMN 100 to be secured to a segment of the fractured bone. More specifically, each of the apertures 280 in the first end portion 120 of the IMN 100 is configured such that when the IMN 100 is inserted into the medullary cavity of a broken bone, a locking screw may be passed through one or more of the apertures 280 to secure or anchor the first end portion 120 to a segment of the fractured bone. The first end portion 120 may include any number of apertures 280. For example, as shown in FIG. 2, the first end portion 120 may include a plurality of apertures 280 disposed at various locations about the first end portion 12 that are arranged and oriented such that locking screws may be inserted through the first end portion 120 at various angles and in various directions. It will be appreciated that the present disclosure is not intended to be limited to any particular number apertures 280 and/or any particular arrangement or orientation of such apertures 280.

In addition to the above, in at least some embodiments, the first end portion 120 of the IMN 100 is configured to be coupled with a handle to facilitate the insertion of the IMN 100 into and the removal of the IMN 100 from the medullary cavity. In such an embodiment, a portion of the passageway 180 disposed in the first end portion 120 may be configured to receive a portion of the handle and to couple the handle to the IMN 100. For example, a portion of the passageway 180 disposed in the first end portion 120 and a portion of the handle may each include threads that when mated together, couple the handle with the IMN 100 (i.e., the handle may be screwed onto the IMN 100). It will be appreciated, however, that other coupling techniques may certainly be used instead, and as such, the present disclosure is not intended to be limited to any particular technique(s).

With continued reference to FIG. 2, the second end portion 140 of the IMN 100 is located at the opposite end of the IMN 100 from the first end portion 120 and is spaced apart from the first end portion 120 by the intermediate portion 160. Like the first end portion 120, the second end portion 140 has a first end 300 and a second end 320, wherein the second end 320 is adjacent to the intermediate portion 160 of the IMN 100 and the first end 300 is spaced from the intermediate portion 160.

The second end portion 140 has a cross-sectional profile (e.g., shape and size (for example, one or more dimensions that extend perpendicularly to the longitudinal axis A, such as height and width)) and cross-sectional area (e.g., cross-sectional surface area) taken in a plane that is perpendicular to the longitudinal axis A (see, for example, FIG. 4 showing the cross-sectional area of the second end portion and a height (h) and a width (w) dimension). In an embodiment, the cross-sectional profile and/or cross-sectional area of the second end portion 140 is constant as the second portion 140 extends from the first end 300 to the second end 320. In other embodiments, however, different parts or sections of the second end portion 140 may have different cross-sectional profiles and/or cross-sectional areas. For example, a first part 340 of the second end portion 140 extending from the first end 300 thereof towards the second end 320 thereof has a constant cross-sectional profile and cross-sectional area, whereas a second part 360 of the second end portion 140 may have a smaller cross-sectional profile and/or cross-sectional area. In one particular example, such as, for example, that shown in FIG. 2, the second part 360 of the second end portion 140 may taper as it extends from the first part 340 towards the second end 320 of the second end portion 140 (i.e., towards the intermediate portion 160 of the IMN 100) such that the cross-sectional profile and cross-sectional area of the second end portion 140 gets progressively smaller as the second part 36 extends toward the second end 320 of the second end portion 140. Accordingly, it will be appreciated that the second end portion may have a single cross-sectional profile and/or area or may have two or more parts or sections thereof that have different cross-sectional profiles and/or areas.

By way of illustration, however, in the embodiment illustrated in FIGS. 1 and 2, the second end portion 140 of the IMN 100 has a cross-sectional profile that is circular in shape, as is shown in FIG. 4, and has a cross-sectional area also shown in FIG. 4. In such an embodiment, the second end portion 140 may a single, constant diameter from the first end 300 to the second end 320 (e.g., a diameter of 9 mm, in an embodiment). In other embodiments, however, different parts or sections of the second end portion 140 may have different outer diameters. For example, in the illustrative embodiment shown in FIG. 2, the first part 340 of the second end portion 140 extending from the first end 30 thereof towards the second end 320 thereof has a constant diameter, whereas the second part 360 of the second end portion 140 tapers as it extends from the first part 340 towards the second end 320 of the second end portion 140 (i.e., towards the intermediate portion 160 of the IMN 100) such that the outer diameter gets progressively smaller as the second part 360 extends toward the second end 320 of the second end portion 140. Accordingly, it will be appreciated that in an embodiment wherein the second end portion 140 has a circular cross-sectional profile, the second end portion 140 may have a single constant outer diameter or may have two or more parts or sections thereof that have different outer diameters.

In addition to the above, and as shown in, for example, FIG. 2, similar to the first end portion 120 described above, the second end portion 140 may also include one or more apertures or holes 280 therein configured to receive locking screws and through which locking screws may pass to allow the second end portion 140 to be secured to a segment of the fractured bone. More specifically, each of the apertures 280 in the second end portion 140 is configured such that when the IMN 100 is inserted into the medullary cavity of a broken bone, a locking screw may be passed through one or more of the apertures 280 to secure or anchor the second end portion 140 to a segment of the fractured bone. The second end portion 140 may include any number of apertures 280. For example, the second end portion 140 may include a plurality of apertures 280 disposed at various locations about the second end portion 140 that are arranged and oriented such that locking screws may be inserted through the second end portion 140 at various angles and in various directions. It will be appreciated that the present disclosure is not intended to be limited to any particular number apertures 280 and/or any particular arrangement or orientation of such apertures 280.

In an embodiment, all or part of the first end portion 120 of the IMN 100 has a first cross-sectional profile and cross-sectional area that are the largest cross-sectional profile and cross-sectional area of the first end portion 120, and all or part of the second end portion 140 of the IMN 10 has a second cross-sectional profile and cross-sectional area that are the largest cross-sectional profile and cross-sectional area of the second end portion 140. The first and second cross-sectional profiles may be equal or substantially equal, or may be different, and the first and second cross-sectional areas may be the equal or substantially equal, or may be different. For example, in an embodiment such as that illustrated in FIG. 2 wherein the first and second end portions 120, 140 both have cross-sectional profiles that are circular in shape, the outer diameter of the first part 240 of the first end portion 120 is equal or substantially equal to the outer diameter of the first part 340 of the second end portion 140, and thus, the first and second cross-sectional profiles are the equal or substantially equal, and the first and second cross-sectional areas are the same (or at least substantially the same), as can be seen in FIG. 3 (first end portion 120) and FIG. 4 (second end portion 140). In one embodiment “substantially equal” means 0-25% of equal, while in another embodiment it means 0-15% of equal, and in yet another embodiment it means 0-10% of equal, and in yet a further embodiment it means 0-5% of equal. In other embodiments, the first and second cross-sectional profiles and/or first and second cross-sectional areas may not be the equal or substantially equal but rather may be different.

As described above, the intermediate portion 160 of the IMN 100 is disposed between the first and second end portions 120, 140 of the IMN 100. The elongate intermediate portion 160 has a cross-sectional profile (e.g., shape and size (for example, one or more dimensions that extend perpendicularly to the longitudinal axis A, such as height and width)) and cross-sectional area (e.g., cross-sectional surface area) taken in a plane that is perpendicular to the longitudinal axis A (see, for example, FIG. 5 showing the cross-sectional area of the second end portion and a height (h) and a width (w) dimension). In an embodiment, the cross-sectional profile and/or cross-sectional area of the intermediate portion 160 is constant as it extends from the first end portion 120 to the second end portion 140. In other embodiments, however, different parts or sections of the intermediate portion 160 may have different cross-sectional profiles and/or cross-sectional areas. In at least one embodiment, at least a portion, and in an embodiment, the entirety, of the intermediate portion 160 has a cross-sectional profile and/or cross-sectional area that is less than the cross-sectional profile and/or cross-sectional area of both the first and second end portions 120, 140. In an embodiment wherein the first and/or second end portion 120, 140 have more than one cross-sectional profile and/or cross-sectional area, at least a portion, and in an embodiment, the entirety, of the intermediate portion 160 has a cross-sectional profile and/or cross-sectional area that is less than at least the largest cross-sectional profile and/or cross-sectional area of the first end portion 120 and the largest cross-sectional profile and/or cross-sectional area of the second end portion 140.

For example, in an embodiment, at least a part or section of the intermediate portion 160 may have a one or more dimensions extending perpendicular to the longitudinal axis A (e.g., the width W and/or height H of the intermediate portion shown in FIG. 5) that is/are less than the largest values of the same dimensions of the first and second portions 120, 140 (e.g., the widths (w) and/or the heights (h) in FIGS. 3 and 4), and as such, the intermediate portion 160, or at least a part or section thereof, will have a cross-sectional profile and cross-sectional area that is less than that of the first and second end portions 120, 140, which is clear when the cross-sectional area of the intermediate portion 160 shown in FIG. 5 is compared with the cross-sectional areas of the first and second end portions 120, 140 shown in FIGS. 3 and 4, respectively.

By way of illustration, in an embodiment, the elongate intermediate portion 160 has a cross-sectional profile that is circular in shape, as is shown in FIG. 5, and has a cross-sectional area also shown in FIG. 5. In such an embodiment, the intermediate portion 160 may have an outer diameter that is constant along its entire length (e.g., a diameter of 7 mm, in an embodiment). In other embodiments, however, different parts or sections of the intermediate portion 160 may have different outer diameters. In any event, at least a part or section, and in an embodiment such as that illustrated in FIG. 2, the entirety, of the intermediate portion 160 has an outer diameter that is smaller than the outer diameters of both the first and second end portions 120, 140. Accordingly, the first end portion 120 has a first outer diameter, the second portion 140 has a second outer diameter, and the intermediate portion 160 has a third outer diameter that is that is smaller than the first and second outer diameters, and thus, the intermediate portion 160 has a cross-sectional profile that is smaller than the cross-sectional profiles of the first and second end portions 120, 140, and a cross-sectional that is smaller than the cross-sectional areas of the first and second portions 120, 140.

The first end portion 120, second end portion 140, and intermediate portion 160 may all be formed or made of the same material(s). Alternatively, one or more of the first end portion 120, second end portion 140, and intermediate portion 160 may be formed of a different material than one or more of the other of the first end portion 120, second end portion 140, and intermediate portion 160. In any instance, materials that may be used to form IMN 100 include, for example and without limitation, stainless steel and titanium or a titanium alloy. It will be appreciated, however, that any material having the suitable strength, durability, and other properties conducive for use as an IMN may be used.

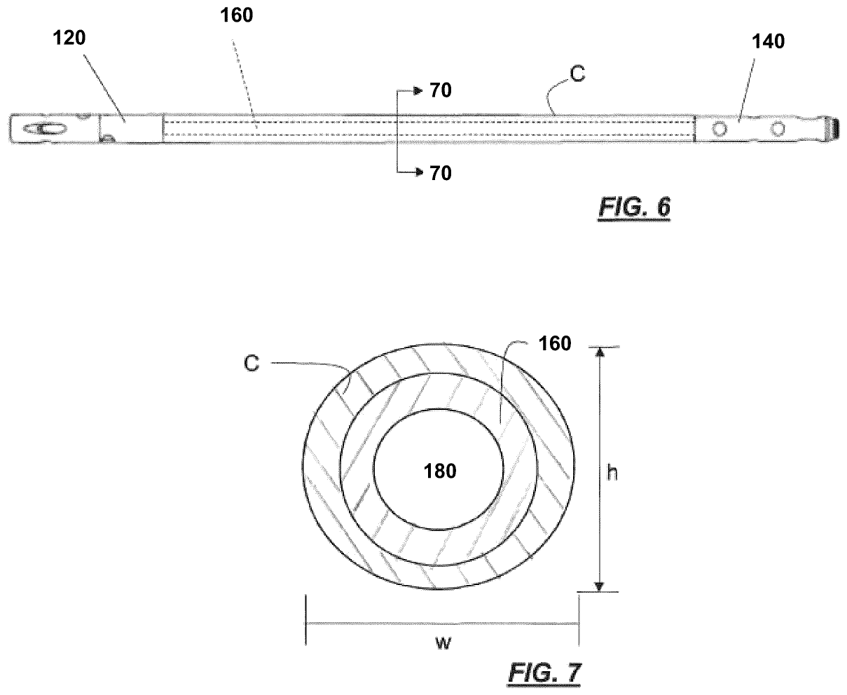

With reference to FIGS. 6, in an embodiment, the IMN 100 comprises an antibiotic or antibiotic-coated IMN. In such an embodiment, the intermediate portion 160 of the IMN 100 and, in at least some embodiments, parts or portions of the first end portion 120 and/or second end portion 140 (e.g., the second part 260 of the first end portion 120 and/or the second part 360 of the second end portion 140) are coated or covered with an antibiotic-loaded bone cement (i.e., antibiotic cement) identified in FIG. 6 by reference character C. In an embodiment, the antibiotic cement comprises a synthetic resin such as, for example, polymethyl methacrylate (PMAA), though other suitable cements may be used instead.

The combination of the intermediate portion 160 and the antibiotic cement C coated thereon has a cross-sectional profile (e.g., shape and size (for example, one or more dimensions that extend perpendicularly to the longitudinal axis A, such as height and width)) and cross-sectional area (e.g., cross-sectional surface area) taken in a plane that is perpendicular to the longitudinal axis A (see, for example, FIG. 7 showing the cross-sectional area of the antibiotic cement-coated intermediate portion 160 and a height (h) and a width (w) dimension). In an embodiment such as that illustrated in FIG. 6, the cross-sectional profile and/or cross-sectional area of the antibiotic cement-coated intermediate portion 160 is equal or substantially equal to that of at least a portion of one or both of the first and second end portions 120, 140 such that there is a smooth transition between the cement-coated intermediate portion 160 and one or both of the first and second end portions 120, 140. In one embodiment “substantially equal” means 0-25% of equal, while in another embodiment it means 0-15% of equal, and in yet another embodiment it means 0-10% of equal, and in yet a further embodiment it means 0-5% of equal

By way of illustration, in an embodiment, each of the first end portion 120, second portion 140, and intermediate portion 16 have cross-sectional profiles that are circular in shape, with the cross-sectional profile and cross-sectional area of the intermediate portion 160 being smaller than that of both of the first and second portions 120, 140 (i.e., the intermediate portion has a smaller outer diameter). In such an embodiment, the outer diameter of the antibiotic cement-coated intermediate portion 160 is equal or substantially equal to one or both of an outer diameter of the first end portion 120 (e.g., the largest outer diameter of the first end portion 120) and an outer diameter of the second end portion 140 (e.g., the largest outer diameter of the second end portion 140) such that there is a smooth transition between the cement coated intermediate portion 160 and one or both of the first and second end portions 120, 140. Accordingly, the first end portion 120 has a first outer diameter, the second portion 140 has a second outer diameter, the intermediate portion 160 has a third outer diameter, and the combination of the intermediate portion 160 and the antibiotic cement comprises a fourth outer diameter that is equal or substantially equal to one or both of the first and second outer diameters. Again, in one embodiment “substantially equal” means 0-25% of equal, while in another embodiment it means 0-15% of equal, and in yet another embodiment it means 0-10% of equal, and in yet a further embodiment it means 0-5% of equal. In other embodiments, the first and second cross-sectional profiles and/or first and second cross-sectional areas may not be equal or substantially equal but rather may be different. In any event, in such an embodiment, the cross-sectional profile and cross-sectional area of the antibiotic cement-coated intermediate portion 160 may be equal or substantially equal to that of at least a portion of one or both of the first end portion 120 and second portion 140 (e.g., the first part 240 of the first end portion 120 and/or the first part 340 of the second end portion 140).

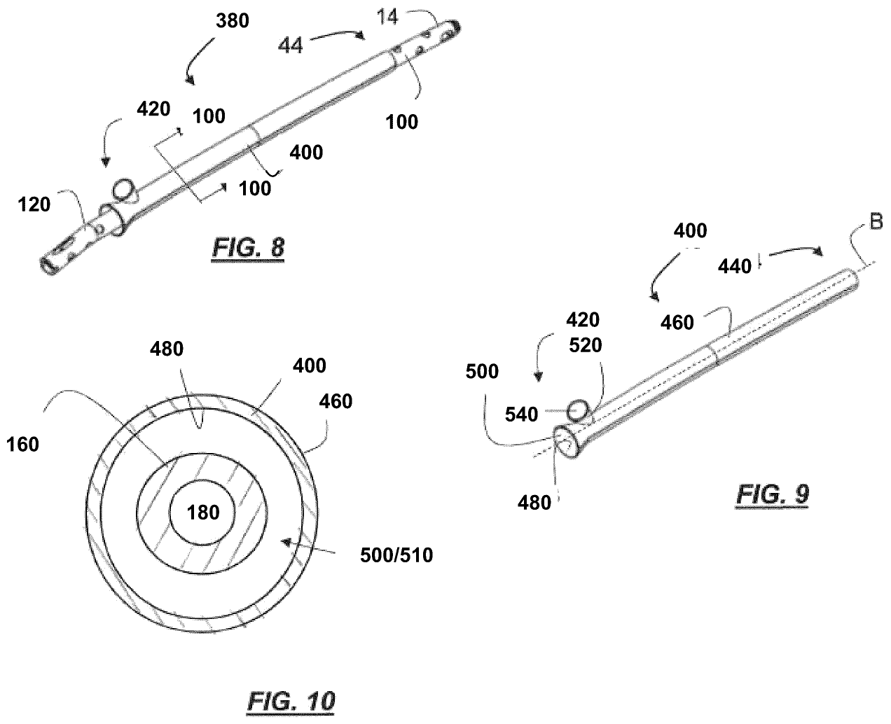

With reference to FIG. 8, while one aspect of the disclosure relates to the IMN 100 described above, another aspect relates to an assembly 38 for use in forming or manufacturing an antibiotic IMN 100, such as, for example, that described above and shown in FIG. 6. In general terms, the assembly 380 comprises an IMN 100 and a sleeve 400, wherein the IMN 100 is inserted into sleeve 400 and/or the sleeve 400 is slid over and onto the IMN 100 such that the sleeve 400 is carried by the IMN 100, and is configured to be removed (e.g., peeled off) following the formation or manufacture of an antibiotic IMN 100.

In the illustrative embodiment depicted in FIG. 8, the IMN 100 of the assembly 38 comprises the IMN 100 described above, the description of which applies here with equal weight and will not be repeated. It will be appreciated, however, that in other embodiments, the assembly 380 may comprise an IMN that differs in one respect or another from the IMN 100 described above, and thus, the present disclosure is not intended to be limited to the assembly 380 including any particular IMN(s). For purposes of illustration, however, the description below will be with respect to an embodiment wherein the assembly 380 includes the IMN 10 described above.

In the illustrative embodiment of the assembly 380 shown in FIG. 8, the sleeve 400 is carried by the IMN 100. As shown in FIGS. 8 and 9, the sleeve 400 has an elongate, tubular-like body having a first end 420, a second end 440 opposite and spaced apart from the first end 420, and a longitudinal axis B (shown in FIG. 9) extending there between. The sleeve 400 also includes an outer surface 460 and an inner surface 480 facing away from the outer surface 460 and defining a sleeve interior 500 that extends between the first and second ends 420, 440, and one or both of the first and second ends 420, 440 of the sleeve comprises an open end to allow the IMN 100 to be inserted into the sleeve 400, and the sleeve interior 500 thereof, in particular.

In an embodiment, the sleeve 400 has a cross-sectional shape taken in a plane that is perpendicular to the longitudinal axis B that is the same as the cross-sectional shape of one or more of the first end portion 120, second portion 140, and intermediate portion 160 of the IMN 100. The sleeve interior 500 may have a cross-sectional profile (e.g., shape and size (for example, one or more dimensions that extend perpendicularly to the longitudinal axis B, such as height and width)) and cross-sectional area (e.g., cross-sectional surface area) taken in a plane that is perpendicular to the longitudinal axis B that is equal or substantially equal to the cross-sectional profile and/or cross-sectional area of at least a portion of each of the first end portion 120 (i.e., the first part 240) and second end portion 140 (i.e., the first portion 340) and larger than the cross-sectional profile and/or cross-sectional area of at least a portion of the intermediate portion 160. In one embodiment “substantially equal” means 0-25% of equal, while in another embodiment it means 0-15% of equal, and in yet another embodiment it means 0-10% of equal, and in yet a further embodiment it means 0-5% of equal.

For example, in at least some embodiments, the sleeve 400 has a circular cross-sectional shape and has an inner diameter and an outer diameter, wherein the inner diameter is equal or substantially equal to both an outer diameter of the first end portion 120 of the IMN 100 and an outer diameter of the second end portion 140 of the IMN 100, and that is larger than the outer diameter of the intermediate portion 150.

When assembled with the IMN 100 as illustrated in FIG. 8, the first end 420 of the sleeve 400 is coupled with the first end portion 120 of the IMN 100, and the second end 440 of the sleeve 400 is coupled with the second end portion 140 of the IMN 100. The first and second ends 420, 440 of the sleeve 400 may be coupled with the first and second end portions 120, 140 of the IMN 100, respectively, in various ways. One way is by an interference or friction fit between the inner surface 480 of the sleeve 400 and outer surfaces of the first and second end portions 120, 140. Another way is that the first and second ends 420, 440 of the sleeve 400 are affixed to the first and second end portions 120, 140, respectively, using a suitable adhesive that is resistant to the maximum curing temperature of the cement. Yet another way is that one or more bands (e.g., rubber band) may be used to clamp or hold each of the ends 420, 440 of the sleeve 400 onto the outer surfaces of the first and second end portions 120, 140 of the IMN 100. While particular examples of have been provided above, any suitable way of coupling the sleeve 400 and IMN 100 together may be used.

In an any event, when the sleeve 400 is assembled with the IMN 100, the sleeve 400 extends between the first and second end portions 120, 140 of the IMN 100 with at least a portion of the sleeve 400 overlapping the intermediate portion 160 of the IMN 100 thereby forming, as shown in FIG. 10, a cavity or void 510 between the inner surface 480 of the sleeve 400 and an outer surface of at least a portion of the intermediate portion 160 that, as will be described below, is configured to be filled with antibiotic cement. In an embodiment, the sleeve 400 also overlaps parts or sections of the first and second end portions 120, 140 of the IMN 100, for example, the second part 260 of the first end portion 120 and the second part 360 of the second end portion 140 shown in FIG. 2.

In addition to the features of the sleeve 400 described above, the sleeve 400 further includes an inlet port 520 located between the first and second ends 420, 440 of the sleeve 400 that includes a throughgoing passageway 540 extending radially from the inner surface 480 of the sleeve 400 through the outer surface 460 of the sleeve 400, and that is in fluid communication with the sleeve interior 500. When the sleeve 400 is assembled with the IMN 100, the throughgoing passageway 540 of the inlet port 520 is in fluid communication with the cavity or void 51 formed between the inner surface 480 of the sleeve 400 and the outer surface of at least a part of the intermediate portion 160 of the IMN 100. The port 520 is configured to allow antibiotic cement to be introduced (e.g., injected) into the cavity or void 510. More specifically, the port 520 is sized and shaped such that it configured to receive a portion of a cement delivery device that is, in turn, configured to introduce or inject the antibiotic cement into the void 510. The inlet port 520 may be located at any suitable location between the first and second ends 420, 440 of the sleeve 400, but in one illustrative embodiment, is located proximate or adjacent to the first end 420. Further, the inlet port 520 may be oriented in any suitable way. In an embodiment such as that illustrated in FIGS. 8 and 9, the inlet port 520 is oriented at a non-zero angle relative to the longitudinal axis B of the sleeve 400, and in particular, at a non-zero angle that is between 1-90° relative to the longitudinal axis B.

As briefly mentioned above, in an embodiment, at least a portion of the interior 500 of the sleeve 400 has a size and shape that substantially matches that of at least a part of the first end portion 120 of the IMN 100 (e.g., the first part 240) and at least part of the second end portion 140 of the IMN 100 (e.g., the first part 340). More particularly, in an embodiment, at least a portion of the interior 500 of the sleeve 400 has a cross-sectional profile and cross-sectional area that substantially matches that of at least a part of the first end portion 120 of the IMN 100 (e.g., the first part 240) and at least part of the second end portion 140 of the IMN 10 (e.g., the first part 340). For example, in an embodiment of the IMN 100 such as that illustrated in FIG. 2, the outer diameter of the first part 240 of the first end portion 120 of the IMN 100 is equal or substantially equal to the outer diameter of the first part 340 of the second end portion 140 of the IMN 100, and the inner diameter of the sleeve 400 is equal or substantially equal to the outer diameters of the first part 240 of the first end portion 120 and the first part 340 of the second end portion 140. Accordingly, when antibiotic cement introduced into the cavity or void 510 between the inner surface 480 of the sleeve 400 and the outer surface of the intermediate portion 160 of the IMN 100 is cured and the sleeve 400 is removed from the IMN 100, the outer diameter of the cement-coated portion of the IMN 100 is equal or substantially equal to the outer diameters of at least parts or sections of the first and second end portions 120, 140 of the IMN 100 so as to form a smooth transition between the cement-coated intermediate portion 16 and one or both of the first and second end portions 120, 140. In one embodiment “substantially equal” means 0-25% of equal, while in another embodiment it means 0-15% of equal, and in yet another embodiment it means 0-10% of equal, and in yet a further embodiment it means 0-5% of equal.

As at least briefly described above, following the introduction of antibiotic cement into the void 510, the assembly is subjected to a known curing process. And once the antibiotic cement is cured, the sleeve 400 is removed from the IMN 100 by, for example, peeling it from the IMN 100 (e.g., the end portions 120, 140 and the antibiotic cement coating the intermediate portion 160). In view of the above, the sleeve 400 must be formed of a material that is both heat resistant, such that it will not melt during an exothermic curing process, and removable (e.g., peelable from the IMN) after manufacture of the antibiotic IMN is complete. Examples of materials that may be used include, but are certainly not limited to, a suitable plastic or flexible polymer, for example, Tygon® tubing commercially available from Saint-Gobain.

In addition to the aspects of the disclosure described above, namely, the IMN 100 and the assembly 380 that comprises an IMN and a sleeve carried by the IMN, yet another aspect of the disclosure relates to a kit for forming or manufacturing an antibiotic IMN that comprises, at least in part, an IMN and a sleeve that is configured to be assembled with the IMN.

In an embodiment, the IMN of the kit comprises the IMN 100 described above, the description of which applies here with equal weight and will not be repeated. It will be appreciated, however, that in other embodiments, the kit may comprise an IMN that differs in one respect or another from the IMN 100 described above, and thus, the present disclosure is not intended to be limited to the kit including any particular IMN(s). For purposes of illustration, however, the description below will be with respect to an embodiment wherein the kit includes the IMN 100 described above.

Similarly, in an embodiment, the sleeve of the kit comprises the sleeve 400 described above, the description of which applies here with equal weight and will not be repeated. It will be appreciated, however, that in other embodiments, the kit may comprise a sleeve that differs in one respect or another from the sleeve 400 described above, and thus, the present disclosure is not intended to be limited to the kit including any particular sleeve(s). For purposes of illustration, however, the description below will be with respect to an embodiment wherein the kit includes the sleeve 400 described above.

The sleeve 400 is configured such that when it is assembled with the IMN 100, the sleeve 400 overlaps at least the intermediate portion 160 of the IMN 10 to form a void or cavity (i.e., the void or cavity 510) between the inner surface 480 of the sleeve 400 and the outer surface of the intermediate portion 160 of the IMN 100 that is configured to be filled with antibiotic cement to form an antibiotic IMN 100. In one embodiment of the kit, the IMN 100 and the sleeve 400 are pre-assembled together. In other embodiments, however, the IMN 100 and sleeve 400 are not pre-assembled but are intended to be assembled/coupled together prior to use.

In an embodiment, in addition to the IMN 100 and the sleeve 400, the kit may further include antibiotic cement. The cement may be pre-mixed or may be in the form of powder that may be mixed when needed and prior to use. In any event, the cement may be used to fill the void between the inner surface 480 of the sleeve 400 and the outer surface of the intermediate portion 160 of the IMN 100 when the sleeve 400 and IMN 10 are assembled together. More specifically, cement may be introduced into the void through the inlet port 520 of the sleeve 400.

It is to be understood that the foregoing is a description of one or more embodiments of the invention. The invention is not limited to the particular embodiment(s) disclosed herein, but rather is defined solely by the claims below. Furthermore, the statements contained in the foregoing description relate to particular embodiments and are not to be construed as limitations on the scope of the invention or on the definition of terms used in the claims, except where a term or phrase is expressly defined above. Various other embodiments and various changes and modifications to the disclosed embodiment(s) will become apparent to those skilled in the art. All such other embodiments, changes, and modifications are intended to come within the scope of the appended claims.

As used in this specification and claims, the terms “e.g.,” “for example,” “for instance,” “such as,” and “like,” and the verbs “comprising,” “having,” “including,” and their other verb forms, when used in conjunction with a listing of one or more components or other items, are each to be construed as open-ended, meaning that the listing is not to be considered as excluding other, additional components or items. Other terms are to be construed using their broadest reasonable meaning unless they are used in a context that requires a different interpretation.

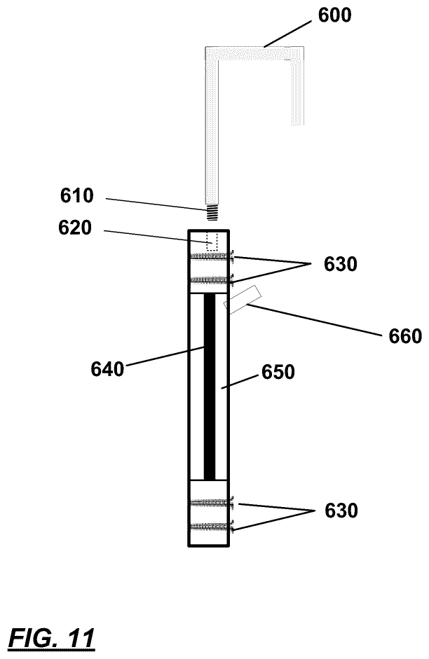

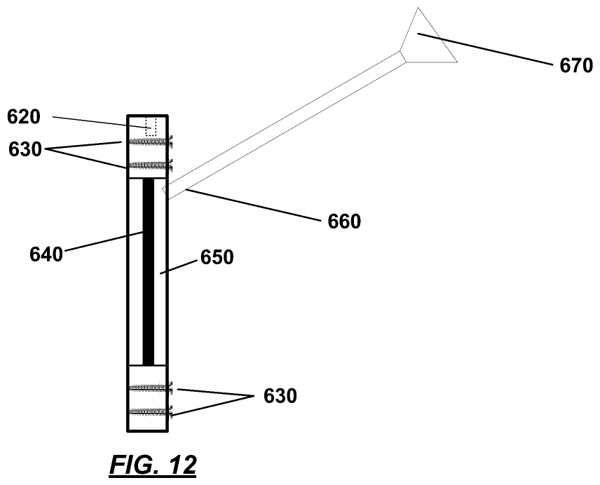

FIG. 11 is a top view diagram illustrating an example of a IMN apparatus with a removably threaded insertion handle and a cement opening in the sleeve.

FIG. 12 is a top view diagram illustrating an example of the IMN apparatus of FIG. 11 with a cement gun connected to the spout opening.

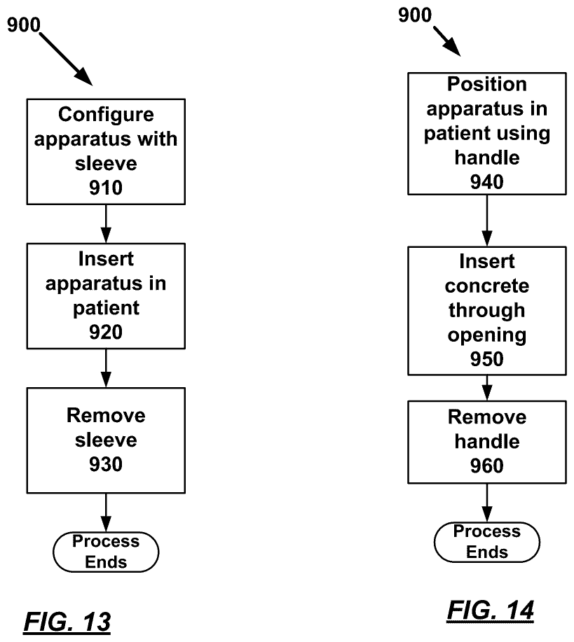

FIG. 13 is a flow chart diagram illustrating an example of a method for applying antibiotic intramedullary nail apparatus.

FIG. 14 is an environmental diagram illustrating an example of a system for applying an antibiotic intramedullary nail apparatus.

1. An apparatus that can include a threaded handle and a removable sleeve for inserting concrete.
